(19) United States
(12) Patent Application Publication
(10) Pub. No.: US 2010/0004345 A1
(43) Pub. Date: Jan. 7, 2010

(54) HIGH GLOSS NON-STICK FILM COATING COMPOSITIONS

(76) Inventors: Charles A. Signorino, Norristown, PA (US); Terry L. Smith, Norristown, PA (US); Stephen Levine, Norristown, PA (US)

Correspondence Address: WILLIAM D. HARE, 66 WITHERSPOON STREET, STE. 1, PMB 317, PRINCETON, NJ, 08542-9944, US

(21) Appl. No.: 12/495,747

(22) Filed: Jun. 30, 2009

Related U.S. Application Data
(60) Provisional application No. 61/077,290, filed on Jul. 1, 2008.

(51) Int. Cl. A61K 47/32

(52) U.S. Cl. 514/772.5

(57) ABSTRACT

The invention generally relates to film coating compositions and processes for film coating pharmaceutical, nutraceutical, and food products. One film coating composition includes a shellac in an aqueous solution; a hydrolyzed starch product; an optional plasticizer; and a coating resin. The coating resin may be one or more of polyvinyl pyrrolidone, polyvinyl alcohol, and poly(vinyl pyrrolidone-vinyl acetate). The shellac, hydrolyzed starch product, plasticizer and coating resin comprise the solids in the composition.

### TECHNICAL FIELD

The field of the invention generally relates to film coating compositions and processes for film coating of pharmaceutical, nutraceutical, and food products.

### BACKGROUND

There is a need to film coat tablets in the pharmaceutical and the nutraceutical industries, as well as a need to coat some foods. However, the coating of tablets to date has often resulted in a low gloss and a disagreeable slimy mouth feel. Many clear unpigmented films do not present a high gloss finish.

One attempt at coating in the past was to use hydroxypropyl methyl cellulose (HPMC), as described in U.S. Pat. No. 5,480,479. Other efforts have been made by others in attempts to improve the appearance and organoleptic properties of coated tablets. For example, see U.S. Pat. No. 4,828,841 to Porter et al., which uses alginates to attempt to improve gloss. Also, attention is directed to U.S. Pat. No. 6,395,298 to Flanagan, which uses the gum gellan to attempt to produce gloss. Neither of these efforts has been completely satisfactory. The coating method disclosed by Porter et al. produces a finish which may be hazy after a build up of material. Flanagan is hard to prepare and apply, and cannot be used at concentrations above 2% gum solids in the coating suspension.

U.S. Pat. No. 4,725,441 discloses coating with maltodextrin (a starch hydrolysate), a plasticizer (glycerol, propylene glycol and polyethylene glycol) and an optional second film forming agent (alginate and propylene glycol alginate). U.S. Pat. No. 5,567,438 discloses shellac coatings in which the shellac is dissolved in a basic aqueous solution to which is added an acidic aqueous solution to form a shellac suspension. The patent further discloses that a plasticizer, such as polyethylene glycol and polypropylene glycol, can be used in the coating formulation.

U.S. Pat. No. 5,059,248 discloses fluid, aqueous pigment dispersions that may be used in film coating compositions for film coating tablets. The dispersions are described as being used with a film coating resin and a plasticizer to formulate film coating compositions. Examples of the film coating resin disclosed are hydroxypropylmethyl cellulose, methyl cellulose, hydroxypropyl cellulose, ethyl cellulose, cellulose acetate phthalate, shellac, and polyvinyl pyrrolidone. Examples of the plasticizer disclosed are propylene glycol, polyethylene glycol, and glycerol.

U.S. Pat. No. 20050031775, the contents of which are incorporated herein, discloses coating systems that include a shellac in an aqueous solution, a hydrolyzed starch product, and a plasticizer. Examples of the hydrolyzed starch product include maltodextrins with a dextrose equivalent (DE) greater than 10 and dried glucose syrups with a DE greater than 20. Examples of the plasticizer include polyethylene glycol and propylene glycol. The coating systems of U.S. 20050031775 provide coatings with a high surface gloss when measured using a TRICOR Surface Analysis System, Model 805A. This publication notes that typical finishes on film coated products have a gloss value of 150, standard clear coat resins may have a gloss value of 200, and the disclosed shellac/hydrolyzed starch coatings may provide a gloss value above 250.

The inventors, however, have determined that the coating systems disclosed in U.S. 20050031775 need to be modified to reduce any potential stickiness or tackiness of the coating and improve the handling properties of the coating system to provide a coating system with improved ease of use and applicability to most, if not all, coating equipment, e.g., fluid bed and pan coaters. If the coating system is too sticky or tacky, it may not be useful in manufacturing coated tablets where the trouble-free operation of equipment is necessary to compete in a competitive industry. To provide a coating system that can be used with commonly available coating equipment and provide the required trouble-free operation of the equipment with the coating system, the inventors attempted a variety of coating resins to reduce stickiness/tackiness and determined that most had the drawback of reducing gloss. However, as described below, PVP, PVPVA and PVA were found to reduce stickiness/tackiness and retain high gloss values.

### SUMMARY

In one general aspect, a film coating composition includes a shellac in an aqueous solution; a hydrolyzed starch product; an optional plasticizer; and a coating resin comprising one or more of polyvinyl pyrrolidone, polyvinyl alcohol, and poly(vinyl pyrrolidone-vinyl acetate). The shellac, hydrolyzed starch product, plasticizer and coating resin make up the solids in the composition.

Embodiments of the film coating composition may include one or more of the following features. For example, the film coating composition may be applied to an edible surface to provide a Tricor reading of gloss that is greater than 200. The film coating composition may be applied to an edible surface to provide a Tricor reading of gloss that is between 200 and 250.

The coating resin may be present in an amount of between approximately 10% and 30% (w/w) of the solids in the composition. The coating resin may be polyvinylpyrrolidone. The coating resin may be poly(vinyl pyrrolidone-vinyl acetate).

The shellac may be present in an amount of between about 10% and 30% (w/w) of the solids in the composition.

The hydrolyzed starch product may be present in an amount of between about 20% and 60% (w/w) of the solids of the composition. The hydrolyzed starch product may be one or both of a maltodextrin and a corn syrup solid. The hydrolyzed starch product may have a dextrose equivalent value of between about 4 and 25, or between 15 and 20.

The plasticizer may be present in an amount of between about 5% and 25% (w/w) of the solids in the composition. The plasticizer may be one or more of triacetin (TA), triethylcitrate (TEC), polyethylene glycol (PEG), propylene glycol (PG), glycerine, glycerol monostearate (GMS), diacetylated monoglyceride (DAM) and polysorbate. The polyethylene glycol may have a molecular weight of 200 to 8000. The polysorbate may be selected from one or more of various polysorbates, such as polysorbate 60 or polysorbate 80. Other polysorbates may be operable.

The composition may be applied to a pharmaceutical product, nutraceutical product, or food product.

The composition may have a viscosity of less than about 400 cP measured at 25° C., and more particularly, less than about 300 cP measured at 25° C.

In another general aspect, a multi-component kit for formulating a film coating composition includes a dry mix component and an aqueous shellac. The dry mix component includes a hydrolyzed starch product; an optional plasticizer; and a coating resin comprising one or more of polyvinyl pyrrolidone, polyvinyl alcohol, and poly(vinyl pyrrolidone-vinyl acetate). The shellac, hydrolyzed starch product, plasticizer and coating resin make up the solids in the composition.

Embodiments of the kit may include one or more of the following features. For example, the film coating composition may be applied to an edible surface to provide a Tricor reading of gloss that is greater than 200. The film coating may be applied to an edible surface to provide a Tricor reading of gloss that is between 200 and 250.

The coating resin may be present in an amount of between approximately 10% and 30% (w/w) of the solids in the composition. The coating resin may be polyvinylpyrrolidone. The coating resin may be poly(vinyl pyrrolidone-vinyl acetate).

The shellac may be present in an amount of between about 10% and 30% (w/w) of the solids in the composition.

The hydrolyzed starch product may be present in an amount of between about 20% and 60% (w/w) of the solids in the composition. The hydrolyzed starch product may be one or both of a maltodextrin and a corn syrup solid. The hydrolyzed starch product may have a dextrose equivalent value of between about 4 and 25, or between 15 and 20.

The plasticizer may be present in an amount of between about 5% and 25% (w/w) of the solids in the composition. The plasticizer may be one or more of triacetin (TA), triethylcitrate (TEC), polyethylene glycol (PEG), propylene glycol (PG), glycerine, glycerol monostearate (GMS), diacetylated monoglyceride (DAM) and polysorbate. The polyethylene glycol may have a molecular weight of 200 to 8000. The polysorbate may be selected from one or more of various polysorbates, such as polysorbate 60 or polysorbate 80. Other polysorbates may be operable.

The composition may be applied to a pharmaceutical product, nutraceutical product, or food product.

The composition made using the kit may have a viscosity of less than about 400 cP measured at 25° C., and more particularly, less than about 300 cP measured at 25° C.

In another general aspect, a method of making a coating mixture includes:

(1) providing a dry mix component, the dry mix component comprising a hydrolyzed starch product, an optional plasticizer, and a coating resin comprising one or more of polyvinyl pyrrolidone, polyvinyl alcohol, and poly(vinyl pyrrolidone-vinyl acetate);

(2) providing an aqueous shellac;

(3) mixing the dry mix component with water to form an aqueous mixture; and

(4) mixing the aqueous mixture with the aqueous shellac to form the coating mixture. The shellac, hydrolyzed starch product, plasticizer and coating resin make up the solids in the composition.

Embodiments of the method may include one or more of the features described above. For example, the method may further include a step of separately providing the hydrolyzed starch product, the plasticizer and the coating resin, and mixing to form the dry mix component.

In the method, the resulting composition may have a viscosity of less than about 400 cP measured at 25° C., and more particularly, less than about 300 cP measured at 25° C.

The details of various embodiments of the invention are set forth in the description below. Other features and advantages of the invention will be apparent from the description and the claims.

### DETAILED DESCRIPTION

The inventors have developed easily applied, high gloss film coating compositions made up of an aqueous shellac, a hydrolyzed starch product, a plasticizer, and a coating resin. The film coating compositions have a high gloss reading on a Tricor glossometer, in particular, greater than 200, and even more particularly, greater than 250. The film coating compositions also provide a surface on the product to which it is applied which is not sticky or tacky, a characteristic that has been identified as occurring in some high gloss film coating compositions. The film coating compositions also are easily prepared, handled and applied because of the compositions' viscosities.

In one implementation, the composition of the film coating composition would consist of the following solids, on a weight/weight basis: shellac, 10-30%; Maltrin, 20-60%; a resin, 5-30%; and a plasticizer, 5-25%.

In another implementation, the composition may be supplied as a kit that includes as a first part, the dry components: Maltrin, the coating resin, and the plasticizer. As a second part, the kit includes the aqueous shellac. In a method of using the kit, the dry components are first mixed with water and to that mixture the aqueous shellac is added. The resulting film coating composition can then be applied to a pharmaceutical product, a nutraceutical product, a food, or a food product.

A shellac deposited out of water may be used as a component of a film forming system to produce a high gloss coating composition. Aqueous based shellac is utilized in the present invention. Aqueous based shellac may be ammoniated shellac to make the shellac soluble in water. The aqueous shellac may be any suitable ammoniated shellac dissolved in water. Such shellacs are commercially available under the trademarks Marcoat 125 and Emcoat 120 available from Emerson Resources, Inc., 600 Markley Street, Norristown, Pa. 19401. Marcoat 125 is an aqueous shellac glaze having 25% shellac in water. Emcoat 120 is an aqueous shellac glaze having 20% shellac in water. All percentages herein, unless otherwise indicated, are expressed as percentages by weight.

Various hydrolyzed starch products may be utilized including maltrodextrins with a dextrose equivalent (DE) between about 4 and 20 and dried glucose syrups, which have a DE above 20. Suitable hydrolyzed starch products are commercially available from Grain Processing Corporation of Muscatine, Iowa under trademarks such as MALTRIN M180, MALTRIN M200 and MALTRIN M250. MALTRIN M180 is a hydrolyzed starch product having a DE of between 16.5 and 19.5, MALTRIN 200 is a hydrolyzed starch product having a DE of between 20 and 23, and MALTRIN M250 is a hydrolyzed starch product having a DE of between 23 and 27. There is a balance between tackiness and gloss considered when selecting the DE of the hydrolyzed starch product. For example, a low DE reduces tackiness while a high DE improves gloss. As such, the inventors have determined that it is preferred to use a hydrolyzed starch product having a DE of 4-25, more preferably a DE of 10-20, and most preferably, a DE of 15-20, in the coating composition of the present invention. However, it is believed that hydrolyzed starch products having a DE greater than the 4-25 range also should be functional.

Compositions of the shellac and hydrolyzed starch products such as MALTRIN M200 may be blended at a ratio of 1:1 and as high as 1:5. Ratios having one part shellac to two or three parts of hydrolyzed starch product give a good gloss and balance shellac's sealing and taste masking with the hydrolyzed starch's solubility and good mouth feel.

The composition may be modified with a suitable plasticizer in an effective amount to create evenness of the coating and reduce stickiness. The plasticizer may be present in an amount of between about 5% and 25% (w/w) of the composition. The plasticizer level is determined by the type of plasticizer used, the ratio of shellac to hydrolyzed starch product, and the level to which the composition may be pigmented. Plasticizers may be selected from the group including triacetin (TA), triethylcitrate (TEC), polyethylene glycol (PEG) (molecular weight (MW) 200-8000), propylene glycol (PG), glycerine, glycerol monostearate (GMS), diacetylated monoglyceride (DAM) and polysorbate 80 (PS80), polysorbate 60, and other molecular weights of polysorbate. As may be seen from the examples hereinafter provided, typical amounts of plasticizer range in the neighborhood of about 5% to 25% (w/w) of the composition, although the level may extend above or below this range. In addition, the use of the plasticizer may be optional for some coating formulations.

The coating resin may be selected from amongst a number of resins. The preferred resins are polyvinylpyrrolidone (PVP), polyvinyl acetate (PVA), and copolymers of (vinylpyrrolidone and vinyl acetate) (copovidone, PVPVA), with PVP being most preferred. One supplier of PVP and PVPVA is ISP Corporation of Wayne, N.J. The molecular weight of the resin also affects the gloss of the coating. A higher molecular weight resin is more preferred that lower molecular weight resins, although both provide high gloss coatings. In particular, it was found that using the higher molecular weight PVP 90K, which has a molecular weight between about 250K and about 360K, gave a higher gloss coating than the lower molecular weight PVP 30K, although both provided high gloss coatings.

The inventors have tested PVP in a molecular weight range of about 1,200 to about 360,000 and consistently achieved a good gloss. It is expected that PVP in a molecular weight outside of this range, and in particular, above this range, will also achieve a good gloss. The ability of PVP, PVPVA, and PVA to provide a high gloss coating is surprising because these resins are of a relatively low molecular weight and not generally promoted for gloss purposes. Moreover, these resins are very different structurally from the high molecular weight gums, such as alginate and gellan, that are typically promoted for gloss. The beneficial advantage provided by the addition of these resins is that they will improve the film quality and still permit gloss readings to be attained in the range of about 200 to 250.

The film coating composition may be further modified with colorants to produce various colors. Further, the film coating composition may be modified with flavors, sweeteners and other organoleptic enhancers without causing adverse effects on the gloss of the coating.

Various colorants may be utilized in the high gloss composition of the present invention including FD&C lakes and dyes, D&C lakes and dyes, titanium dioxide, iron oxides, pigments deposited on mica powder (micanized pigments) and other natural colorants. Colorants may be added to the system at up to 20% to 40% of the combined weight of the shellac and hydrolyzed starch product.

The edible film coating composition of the present invention provides surprisingly high gloss. Using the TRICOR Surface Analysis System, Model 805A, to measure surface gloss, typical finishes on film coated products give readings up to 150. With the standard clear coat of resins currently used, the gloss values may be raised to approach 200. With the film forming composition of the present invention, which includes an aqueous based shellac, a hydrolyzed starch product, an optional plasticizer and a coating resin, gloss values above 200 and, more particularly, approaching 250 or greater may be readily achieved while also providing an easily handled and non-sticky product. In particular, the viscosity is less than the viscosity of some of the prior art film coating compositions. This reduction in viscosity improves the ease of handling and reduces the stickiness and tackiness of the coating.

The inventors have found that a suitable gloss was found when a weight buildup of between about 0.25% and 3% was used. However, a strict correlation between weight buildup and gloss has not been found and appears to be dependent on the particular formulation and excipients used. Thus, a minor amount of experimentation, based on varying the amount of coating buildup, should suffice to optimize the gloss. It should be noted that while a range of about 0.25% to 3% is recommended, a build up above or below this range also may provide a suitable gloss.

The following are some examples to illustrate the present invention. In each of the examples illustrated below, the aqueous shellac was obtained from Marcoat 125 which is a 25% solids solution, that is, it contains 25% shellac. In each of the following example formulations, the amount of each component present is based on a total of 100 parts by weight. In each example where the quantity of Marcoat 125 is indicated, the shellac present is 25% of the quantity indicated, since Marcoat 125 is a 25% shellac solution. Thus, 10 grams of Marcoat 125 includes 2.5 grams of shellac. It should be noted that although an aqueous shellac with 25% solids is used in the example, other aqueous-based shellacs also would be operable, including an aqueous shellac with 20% solids.

In the examples, the following measurements are used:

Tricor Gloss Reading—a measure of gloss of a surface in which an increased value indicates increased gloss; measured using a TRICOR Surface Analysis System.

Orange Peel—a subjective observation of smoothness and levelness of a surface. Using this measure one can determine the extent to which the droplets of the coating become smooth or level on the surface of the tablet.

Rashy—a subjective observation of the unevenness of the coating surface, in which 1 is least rashy and 5 is most rashy.

Overall—an overall, subjective measure of the surface of the coated dosage form in which 1 is excellent and 5 is worst case.

### EXAMPLE 1

### Polyvinyl Alcohol-Based Film Coating Kit

Part A. Dry Mix Component

| | | | Gm solid/ | | % solid | |
|---|---|---|---|---|---|---|
| Component | gm/batch | % w/w | batch | % solids | w/w | % polymer |
| Maltrin 150 | 20.00 | 43.22 | 20.00 | 100.00 | 100.00 | 100.00 |
| (hydrolyzed | | | | | | |
| starch product) | | | | | | |
| PVA (coating | 20.00 | 43.22 | 20.00 | 100.00 | 100.00 | 100.00 |
| resin) | | | | | | |
| PS80 | 1.00 | 2.16 | 1.00 | 100.00 | 5.00 | 5.00 |
| (plasticizer) | | | | | | |
| PEG 3350 | 5.28 | 11.41 | 5.28 | 100.00 | 26.40 | 26.40 |
| (plasticizer) | | | | | | |
| Total Blend | 46.28 | 100.0 | 46.28 | 100.00 | 100.00 | — |

Procedure:

1. The Maltrin 150 (hydrolyzed starch product, maltodextrin with a starch equivalent of between 15 and 17) (source, Grain Processing Corporation (GPC) of Muscatine, Iowa), polyvinyl alcohol (PVA) (source, Fisher Healthcare of Houston, Tex., 100,000 molecular weight, 87% soluble), polysorbate 80 (PS80) (source, Unigema, B-Everberg), polyethylene glycol (PEG) 3350 (source, Clariant Corporation of Somerville, N.J.) were mixed using a blender or beaker to form a homogeneous dry mix.

2. The dry mix was packaged and stored in a double sealed, zip-tied bag.

Part B. Liquid Components

Marcoat 125 (shellac, 25% solids) (source, Emerson Resources, Norristown, Pa.)

Deionized Water

Coating composition:

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The dry mix component and deionized water were mixed to form a homogenous suspension free of clumps of solid material. The Marcoat 125 was added with mixing until a smooth lump free suspension was achieved.

Coating process:

To test the coating composition for gloss and overall characteristics, 2 kilograms of placebo tablets were placed in a 15 inch O'Hara coating pan and operated with an inlet air temperature of between 59 and 64° C., an air flow rate of between 148 and 151, an atomizer pressure of 20 psi, an RPM of between 15 and 16, and a coating flow rate of approximately 15 grams per minute. For applying the coating, a Schlick gun was used with a distance of between 5.5 and 6 inches from the bed The coating process was performed for approximately 30 minutes during which 471 grams of the coating material was applied, which corresponds to a weight gain of 1%. The coated tablets were tested for gloss and surface characteristics. The results are illustrated in Table 1.

TABLE 1
Polyvinyl Alcohol Coating Tablet Characteristics

| Weight Gain | Tricor Gloss | Orange Peel | | | |
|---|---|---|---|---|---|
| (%) | Reading | Frequency | Amplitude | Rashy | Overall |
| 0.25 | 213 | 0.995 | 18015 | 3 | 3 |
| 0.50 | 218 | 0.995 | 20008 | 3 | 3 |
| 0.75 | 223 | 0.995 | 20879 | 3 | 3 |
| 1.00 | 221 | 0.995 | 20756 | 3 | 3 |

The results listed in Table 1 indicate that the coating provides a high gloss coating that has a Tricor gloss reading of between 213 and 223.

Examples 2-5 illustrate the gloss differences between different povidone compounds that differ by molecular weight.

### EXAMPLE 2

### Plasdone K-12-Based Film Coating Kit

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 20.00 | 44.17 | 20.00 | 100.00 | 100.00 | 100.00 |
| (hydrolyzed | | | | | | |
| starch | | | | | | |
| product) | | | | | | |
| Plasdone K-12 | 20.00 | 44.17 | 20.00 | 100.00 | 100.00 | 100.00 |
| (coating resin) | | | | | | |
| PEG 3350 | 5.28 | 11.66 | 5.28 | 100.00 | 26.40 | 26.40 |
| (plasticizer) | | | | | | |
| Total Blend | 45.28 | 100.0 | 45.28 | 100.00 | 100.00 | — |

Procedure: The Maltrin 150 (hydrolyzed starch product, maltodextrin with a starch equivalent of between 15 and 17) (source, Grain Processing Corporation of Muscatine, Iowa), plasdone K-12 (N-vinyl-2-pyrrolidone, povidone, 4,000 molecular 15 weight) (source, ISP Corporation of Wayne, N.J.), polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson of Norristown, Pa.)

Deionized Water

Coating composition

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous starch) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 62 and 66° C., an atomizer pressure of 25 psi, an RPM of approximately 16, and a coating flow rate of between approximately 13 and 21 grams per minute. For applying the coating, a Schlick gun was used with a distance of between 5.5 and 6 inches from the bed The coating process was performed for approximately 25 minutes during which 471 grams of the coating material was applied, which corresponds to a weight gain of 1%. The coated tablets were tested for gloss and surface characteristics. The results are illustrated in Table 2.

TABLE 2
Plasdone K-12 Coating Tablet Characteristics

| Weight Gain | Tricor Gloss | Orange Peel | | |
|---|---|---|---|---|
| (%) | Reading | Frequency | Amplitude | Overall |
| 0.25 | 230 | 0.996 | 20019 | 3 |
| 0.50 | 221 | 0.996 | 19970 | 3 |
| 0.75 | 226 | 0.996 | 22175 | 4 |
| 1.00 | 214 | 0.996 | 20631 | 4 |

Viscosity Measurement: seven seconds in a Zahn cup.

The tablets were described as being rashy at 1.00% weight gain.

### EXAMPLE 3

### Plasdone K-17-Based Film Coating Kit

Part A. Dry Mix Component

| | | | gm solid/ | | % solid | |
|---|---|---|---|---|---|---|
| Component | Gm/batch | % w/w | batch | % solids | w/w | % polymer |
| Maltrin 150 | 20.00 | 44.17 | 20.00 | 100.00 | 100.00 | 100.00 |
| (hydrolyzed | | | | | | |
| starch | | | | | | |
| product) | | | | | | |
| Plasdone K-17 | 20.00 | 44.17 | 20.00 | 100.00 | 100.00 | 100.00 |
| (coating resin) | | | | | | |
| PEG 3350 | 5.28 | 11.66 | 5.28 | 100.00 | 26.40 | 26.40 |
| (plasticizer) | | | | | | |
| Total Blend | 45.28 | 100.0 | 45.28 | 100.00 | 100.00 | — |

Procedure: The Maltrin 150 (hydrolyzed starch product, maltodextrin with a starch equivalent of between 15 and 17) (source, Grain Processing Corporation of Muscatine, Iowa), plasdone K-17 (N-vinyl-2-pyrrolidone, povidone, 10,000 molecular weight) (source, ISP Corporation of Wayne, N.J.), polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson Resources, Norristown, Pa.)

Deionized Water

Coating composition:

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 65 and 68° C., an air flow rate of between 148 and 152, an atomizer pressure of 25 psi, an RPM of 16, and a coating flow rate of between approximately 12 and 16 grams per minute. For applying the coating, a Schlick gun was used with a distance of between 5.5 and 6 inches from the bed The coating process was performed for approximately 30 minutes during which 471 grams of the coating material was applied, which corresponds to a weight gain of 1%. The coated tablets were tested for gloss and surface characteristics. The results are illustrated in Table 3.

TABLE 3
Plasdone K-17 Coating Tablet Characteristics

| Weight Gain | Tricor Gloss | Orange Peel | | |
|---|---|---|---|---|
| (%) | Reading | Frequency | Amplitude | Overall |
| 0.25 | 222 | 0.996 | 18340 | 3 |
| 0.50 | 215 | 0.996 | 18334 | 3 |
| 0.75 | 216 | 0.994 | 19251 | 4 |
| 1.00 | 215 | 0.995 | 18894 | 4 |

Viscosity Measurement: seven seconds in a Zahn cup.

### EXAMPLE 4

### Plasdone K-90-Based Film Coating Kit

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 20.00 | 44.17 | 20.00 | 100.00 | 100.00 | 100.00 |
| (hydrolyzed | | | | | | |
| starch | | | | | | |
| product) | | | | | | |
| Plasdone K-90 | 20.00 | 44.17 | 20.00 | 100.00 | 100.00 | 100.00 |
| (coating resin) | | | | | | |
| PEG 3350 | 5.28 | 11.66 | 5.28 | 100.00 | 26.40 | 26.40 |
| (plasticizer) | | | | | | |
| Total Blend | 45.28 | 100.0 | 45.28 | 100.00 | 100.00 | — |

Procedure: The Maltrin 150 (hydrolyzed starch product, maltodextrin with a starch equivalent of between 15 and 17) (source, Grain Processing Corporation (GPC) of Muscatine, Iowa), plasdone K-90 (N-vinyl-2-pyrrolidone, povidone, 1,300,000 molecular weight) (source, ISP Corporation of Wayne, N.J.), and polyethylene glycol (PEG) 3350 (source, Clariant Corporation of Somerville, N.J.) were mixed, packaged an stored as in Example 1.

Part B. Liquid Components

Marcoat 125 (shellac, 25% solids) (source, Emerson Resources, Norristown, Pa.)

Deionized Water

Coating composition:

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 58 and 66° C., an air flow rate of between 150 and 152, an atomizer pressure of 25 psi, an RPM of approximately 16, and a coating flow rate of between approximately 15 and 20 grams per minute. For applying the coating, a Schlick gun was used with a distance of between 5.5 and 6 inches from the bed The coating process was performed for approximately 25 minutes during which 471 grams of the coating material was applied, which corresponds to a weight gain of 1%. The coated tablets were tested for gloss and surface characteristics. The results are illustrated in Table 4.

TABLE 4
Polysorbate K-90 Coating Tablet Characteristics

| Weight Gain | Tricor Gloss | Orange Peel | | |
|---|---|---|---|---|
| (%) | Reading | Frequency | Amplitude | Overall |
| 0.25 | 224 | 0.996 | 20656 | 4 |
| 0.50 | 231 | 0.996 | 22174 | 3 |
| 0.75 | 226 | 0.997 | 22754 | 4 |
| 1.00 | 237 | 0.996 | 23185 | 4 |

Viscosity Measurement: seven seconds in a Zahn cup.

The tablets were described as being rashy at 0.25% weight gain and having an orange peel surface at 0.75 and 1.00% weight gain.

Examples 2-4 above illustrate the improvement in gloss with the use of a higher weight PVP. The gloss readings indicate a trend towards increasing gloss with the use of the higher molecular weight PVP.

Examples 5-8 illustrate the differences in the coating when using Maltrin of different dextrin equivalents.

### EXAMPLE 5

### Plasdone K-90-Based Film Coating Kit using Maltrin 150

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 20.00 | 43.22 | 20.00 | 100.00 | 100.00 | 100.00 |
| (hydrolyzed | | | | | | |
| starch | | | | | | |
| product) | | | | | | |
| Plasdone K-90 | 20.00 | 43.22 | 20.00 | 100.00 | 100.00 | 100.00 |
| (coating resin) | | | | | | |
| PS80 | 1.00 | 2.16 | 1.00 | 100.00 | 5.00 | 5.00 |
| (plasticizer) | | | | | | |
| PEG 3350 | 5.28 | 11.41 | 5.28 | 100.00 | 26.40 | 26.40 |
| (plasticizer) | | | | | | |
| Total Blend | 46.28 | 100.0 | 46.28 | 100.00 | 100.00 | — |

Procedure: The Maltrin 150 (hydrolyzed starch product, maltodextrin with a starch equivalent of between 15 and 17) (source, Grain Processing Corporation (GPC) of Muscatine, Iowa), plasdone K-90 (N-vinyl-2-pyrrolidone, povidone, 1,300,000 molecular weight) (source, ISP Corporation of Wayne, N.J.), polysorbate 80 (PS80) (source, Unigema, B-Everberg), and polyethylene glycol (PEG) 3350 (source, Clariant Corp. of Somerville, N.J.) were mixed, packaged and stored as in Example 1.

Part B. Liquid Components

Marcoat 125 (shellac, 25% solids) (source, Emerson of Norristown, Pa.)

Deionized Water

Coating composition:

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 59 and 60° C., an air flow rate of between 148 and 152, an atomizer pressure of 25 psi, an RPM of approximately 16, and a coating flow rate of between approximately 14 and 19 grams per minute. For applying the coating, a Schlick gun was used with a distance of between 5.5 and 6 inches from the bed The coating process was performed for approximately 27 minutes during which 471 grams of the coating material was applied, which corresponds to a weight gain of 1%. The coated tablets were tested for gloss and surface characteristics. The results are illustrated in Table 5.

TABLE 5
Maltrin 150 Coating Tablet Characteristics

| Weight Gain | Tricor Gloss | Orange Peel | | |
|---|---|---|---|---|
| (%) | Reading | Frequency | Amplitude | Overall |
| 0.25 | 235 | 0.996 | 20911 | 2 |
| 0.50 | 238 | 0.996 | 20968 | 2 |
| 0.75 | 239 | 0.997 | 21666 | 2 |
| 1.00 | 247 | 0.996 | 22785 | 1.5 |

### EXAMPLE 6

### Plasdone K-90-Based Film Coating Kit using Maltrin 180

Part A. Dry Mix Component

| | | | gm solid/ | | % solid | |
|---|---|---|---|---|---|---|
| Component | gm/batch | % w/w | batch | % solids | w/w | % polymer |
| Maltrin 180 | 20.00 | 43.22 | 20.00 | 100.00 | 100.00 | 100.00 |
| (hydrolyzed | | | | | | |
| starch product) | | | | | | |
| Plasdone K-90 | 20.00 | 43.22 | 20.00 | 100.00 | 100.00 | 100.00 |
| (coating resin) | | | | | | |
| PS80 | 1.00 | 2.16 | 1.00 | 100.00 | 5.00 | 5.00 |
| (plasticizer) | | | | | | |
| PEG 3350 | 5.28 | 11.41 | 5.28 | 100.00 | 26.40 | 26.40 |
| (plasticizer) | | | | | | |
| Total Blend | 46.28 | 100.0 | 46.28 | 100.00 | 100.00 | — |

Procedure: The Maltrin 180 (hydrolyzed starch product, maltodextrin with a dextrose equivalent of between 16.5 and 19) (source, Grain Processing Corporation (GPC) of Muscatine, Iowa), plasdone K-90 (N-vinyl-2-pyrrolidone, povidone, 1,300,000 molecular weight) (source, ISP Corporation of Wayne, N.J.), polysorbate 80 (PS80) (source, Unigema, B-Everberg), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson of Norristown, Pa.)

Deionized Water

Coating composition:

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 51 and 62° C., an air flow rate of between 149 and 152, an atomizer pressure of 25 psi, an RPM of between approximately 14 and 17, and a coating flow rate of between approximately 12 and 19 grams per minute. For applying the coating, a Schlick gun was used with a distance of between 5.5 and 6 inches from the bed The coating process was performed for approximately 26.5 minutes during which 471 grams of the coating material was applied, which corresponds to a weight gain of 1%. The coated tablets were tested for gloss and surface characteristics. The results are illustrated in Table 6.

TABLE 6
Maltrin 180 Coating Tablet Characteristics

| Weight Gain | Tricor Gloss | Orange Peel | | | |
|---|---|---|---|---|---|
| (%) | Reading | Frequency | Amplitude | Rash | Overall |
| 0.25 | 230 | 0.995 | 22662 | 1 | 1 |
| 0.50 | 238 | 0.996 | 21680 | 1 | 1 |
| 0.75 | 237 | 0.996 | 22056 | 1 | 1 |
| 1.00 | 260 | 0.996 | 25200 | 1 | 1 |

### EXAMPLE 7

### Plasdone K-90-Based Film Coating Kit using Maltrin 200

Part A. Dry Mix Component

| | | | gm solid/ | | % solid | |
|---|---|---|---|---|---|---|
| Component | gm/batch | % w/w | batch | % solids | w/w | % polymer |
| Maltrin 200 | 20.00 | 43.22 | 20.00 | 100.00 | 100.00 | 100.00 |
| (hydrolyzed | | | | | | |
| starch | | | | | | |
| product) | | | | | | |
| Plasdone K-90 | 20.00 | 43.22 | 20.00 | 100.00 | 100.00 | 100.00 |
| (coating resin) | | | | | | |
| PS80 | 1.00 | 2.16 | 1.00 | 100.00 | 5.00 | 5.00 |
| (plasticizer) | | | | | | |
| PEG 3350 | 5.28 | 11.41 | 5.28 | 100.00 | 26.40 | 26.40 |
| (plasticizer) | | | | | | |
| Total Blend | 46.28 | 100.0 | 46.28 | 100.00 | 100.00 | — |

Procedure: The Maltrin 200 (hydrolyzed starch product, maltodextrin with a dextrose equivalent of between 20 and 23) (source, Grain Processing Corporation (GPC) of Muscatine, Iowa), plasdone K-90 (N-vinyl-2-pyrrolidone, povidone, 1,300,000 molecular weight) (source, ISP Corporation of Wayne, N.J.), polysorbate 80 (PS80) (source, Unigema, B-Everberg), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson of Norristown, Pa.)

Deionized Water

Coating composition:

| Component | gm/batch | % w/w | gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 60 and 61° C., an air flow rate of between 148 and 153, an atomizer pressure of 25 psi, an RPM of approximately 16, and a coating flow rate of between approximately 12 and 16 grams per minute. For applying the coating, a Schlick gun was used with a distance of between 5.5 and 6 inches from the bed The coating process was performed for approximately 33 minutes during which 471 grams of the coating material was applied, which corresponds to a weight gain of 1%. The coated tablets were tested for gloss and surface characteristics. The results are illustrated in Table 7.

TABLE 7
Maltrin 200 Coating Tablet Characteristics

| Weight Gain | Tricor Gloss | Orange Peel | | | |
|---|---|---|---|---|---|
| (%) | Reading | Frequency | Amplitude | Rash | Overall |
| 0.25 | 237 | 0.996 | 24746 | 1 | 1 |
| 0.50 | 250 | 0.996 | 23361 | 1 | 1 |
| 0.75 | 257 | 0.996 | 24792 | 1 | 1 |
| 1.00 | 261 | 0.996 | 24705 | 1 | 1 |

### EXAMPLE 8

### Plasdone K-90-Based Film Coating Kit using Maltrin 250

Part A. Dry Mix Component

| | | | gm solid/ | | % solid | |
|---|---|---|---|---|---|---|
| Component | gm/batch | % w/w | batch | % solids | w/w | % polymer |
| Maltrin 250 | 20.00 | 43.22 | 20.00 | 100.00 | 100.00 | 100.00 |
| (hydrolyzed | | | | | | |
| starch | | | | | | |
| product) | | | | | | |
| Plasdone K-90 | 20.00 | 43.22 | 20.00 | 100.00 | 100.00 | 100.00 |
| (coating resin) | | | | | | |
| PS80 | 1.00 | 2.16 | 1.00 | 100.00 | 5.00 | 5.00 |
| (plasticizer) | | | | | | |
| PEG 3350 | 5.28 | 11.41 | 5.28 | 100.00 | 26.40 | 26.40 |
| (plasticizer) | | | | | | |
| Total Blend | 46.28 | 100.0 | 46.28 | 100.00 | 100.00 | — |

Procedure: The Maltrin 250 (hydrolyzed starch product, maltodextrin with a dextrose equivalent of between 23 and 27) (source, Grain Processing Corporation (GPC) of Muscatine, Iowa), plasdone K-90 (N-vinyl-2-pyrrolidone, povidone, 1,300,000 molecular weight) (source, ISP Corporation of Wayne, N.J.), polysorbate 80 (PS80) (source, Unigema, B-Everberg), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson Resources, Norristown, Pa.)

Deionized Water

Coating composition:

| Component | Gm/batch | % w/w | Gm solid/batch | % solid |
|---|---|---|---|---|
| Dry Mix | 42.00 | 3.84 | 42.00 | 100.00 |
| Marcoat 125 | 51.00 | 4.66 | 12.75 | 25.00 |
| (aqueous shellac) | | | | |
| Deionized water | 1002.00 | 91.51 | 0.00 | 0.00 |
| Total Coating | 1095.00 | 100.0 | 54.75 | 5.00 |
| System | | | | |

Procedure: The coating composition was prepared as in Example 1.

Coating process:

To test the coating composition for gloss and overall characteristics, 2 kilograms of placebo tablets were placed in a 15 inch O'Hara coating pan and operated with an inlet air temperature of between 50 and 64° C., an air flow rate of between 150 and 152, an atomizer pressure of 25 psi, an RPM of approximately 16, and a coating flow rate of between approximately 12 and 16 grams per minute. For applying the coating, a Schlick gun was used with a distance of between 5.5 and 6 inches from the bed The coating process was performed for approximately 32.5 minutes during which 471 grams of the coating material was applied, which corresponds to a weight gain of 1%. The coated tablets were tested for gloss and surface characteristics. The results are illustrated in Table 8.

TABLE 8
Maltrin 250 Coating Tablet Characteristics

| Weight Gain | Tricor Gloss | Orange Peel | | | |
|---|---|---|---|---|---|
| (%) | Reading | Frequency | Amplitude | Rash | Overall |
| 0.25 | 240 | 0.995 | 21224 | 1 | 1 |
| 0.50 | 245 | 0.996 | 21930 | 1 | 1 |
| 0.75 | 242 | 0.996 | 22341 | 1 | 1 |
| 1.00 | 255 | 0.996 | 23825 | 1 | 1 |

Examples 5-8 illustrate that an increase in coating weight from 0.25% to 1% generally increases the gloss value, as does an increase in the dextrose equivalents of the maltodextrin. The following examples, Examples 9-11, illustrate the use of the coating formulations in a column coater.

### EXAMPLE 9

### Plasdone K-90-Based Film Coating Kit using a Column Coater

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 3.00 | 42.86 | 3.00 | 100.00 | 100.00 | 100.00 |
| (hydrolyzed | | | | | | |
| starch | | | | | | |
| product) | | | | | | |
| Plasdone K-90 | 3.00 | 42.86 | 3.00 | 100.00 | 100.00 | 100.00 |
| (coating resin) | | | | | | |
| PEG 3350 | 1.00 | 14.29 | 1.00 | 100.00 | 33.33 | 33.33 |
| (plasticizer) | | | | | | |
| Total Blend | 7.00 | 100.0 | 7.00 | 100.00 | 100.00 | — |

Procedure: The Maltrin 150 (hydrolyzed starch product, maltodextrin with a dextrose equivalent of between 15 and 17) (source, Grain Processing Corporation (GPC) of Muscatine, Iowa), plasdone K-90 (N-vinyl-2-pyrrolidone, povidone, 1,300,000 molecular weight) (source, ISP Corporation of Wayne, N.J.), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson Resources, Norristown, Pa.)

Deionized Water

Coating composition

| Component | Gm/batch | % w/w | Gm solid/batch | % solid |
|---|---|---|---|---|
| Dry Mix | 7.00 | 3.50 | 7.00 | 100.00 |
| Marcoat 125 | 12.00 | 6.00 | 3.00 | 25.00 |
| (aqueous shellac) | | | | |
| Deionized water | 181.00 | 90.50 | 0.00 | 0.00 |
| Total Coating | 200.00 | 100.0 | 10.00 | 5.00 |
| System | | | | |

Procedure: The coating composition was prepared as in Example 1.

Coating process:

To test the coating composition for gloss and overall characteristics, 400 grams of placebo tablets were placed in a column coater and operated with an inlet air temperature of between 48 and 62° C., an atomizer pressure of 25 psi, and a coating flow rate of between approximately 5.75 and 8.5 grams per minute. The coating process was performed for approximately 16 minutes during which 107 grams of the coating material was applied, which corresponds to a weight gain of 1%. The coated tablets were tested for gloss and surface characteristics. The results are illustrated in Table 9.

TABLE 9
Coating Column Tablet Characteristics

| Weight Gain | Tricor Gloss | Orange Peel | | | |
|---|---|---|---|---|---|
| (%) | Reading | Frequency | Amplitude | Rash | Overall |
| 0.25 | 226 | 0.996 | 25440 | 4 | 4 |
| 0.50 | 234 | 0.997 | 30016 | 4 | 4 |
| 0.75 | 228 | 0.998 | 26236 | 4 | 4 |
| 1.00 | 242 | 0.998 | 33625 | 4 | 4 |

### EXAMPLE 10

### Plasdone K-17-Based Film Coating Kit using a Column Coater

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 3.00 | 42.86 | 3.00 | 100.00 | 100.00 | 100.00 |
| (hydrolyzed | | | | | | |
| starch | | | | | | |
| product) | | | | | | |
| Plasdone K-17 | 3.00 | 42.86 | 3.00 | 100.00 | 100.00 | 100.00 |
| (coating resin) | | | | | | |
| PEG 3350 | 1.00 | 14.29 | 1.00 | 100.00 | 33.33 | 33.33 |
| (plasticizer) | | | | | | |
| Total Blend | 7.00 | 100.0 | 7.00 | 100.00 | 100.00 | — |

Procedure: The Maltrin 150 (hydrolyzed starch product, maltodextrin with a dextrose equivalent of between 15 and 17) (source, Grain Processing Corporation (GPC) of Muscatine, Iowa), plasdone K-17 (N-vinyl-2-pyrrolidone, povidone, 1,700 molecular weight) (source, ISP Corporation of Wayne, N.J.), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson Resources, Norristown, Pa.)

Deionized Water

Coating composition:

| Component | Gm/batch | % w/w | Gm solid/batch | % solid |
|---|---|---|---|---|
| Dry Mix | 7.00 | 3.50 | 7.00 | 100.00 |
| Marcoat 125 | 12.00 | 6.00 | 3.00 | 25.00 |
| (aqueous shellac) | | | | |
| Deionized water | 181.00 | 90.50 | 0.00 | 0.00 |
| Total Coating | 200.00 | 100.0 | 10.00 | 5.00 |
| System | | | | |

Procedure: The coating composition was prepared as in Example 1.

Coating process:

To test the coating composition for gloss and overall characteristics, 400 grams of placebo tablets were placed in a column coater and operated with an atomizer pressure of 25 psi and a coating flow rate of between approximately 6.6 and 9.3 grams per minute. The coating process was performed for approximately 14 minutes during which 107 grams of the coating material was applied, which corresponds to a weight gain of 1%. The coated tablets were tested for gloss and surface characteristics. The results are illustrated in Table 10.

TABLE 10
Coating Column Tablet Characteristics

| Weight Gain | Tricor Gloss | Orange Peel | | | |
|---|---|---|---|---|---|
| (%) | Reading | Frequency | Amplitude | Rash | Overall |
| 0.25 | 250 | 0.996 | 23909 | 1 | 1 |
| 0.50 | 245 | 0.996 | 22021 | 1 | 1.5 |
| 0.75 | 230 | 0.996 | 22840 | 1.5 | 1.5 |
| 1.00 | 224 | 0.996 | 23571 | 2.5 | 2 |

The results illustrated in Table 10 can be compared to those of Table 3 associated with Example 3, above, in which a pan coater is used with very similar amounts of the same components. The pan coater had a decrease in gloss in comparison to the column coater. In particular, at 0.25% weight gain, the column coater gave a gloss value of 250 versus a gloss value of 222 for the pan coater; at 0.5% weight gain the values were 245 versus 215; at 0.75% weight gain the values were 230 versus 216; and at 1.0% weight gain the values were 224 versus 215. Moreover, the pan coater gave a better overall appearance with scores between 1 and 2 versus scores of 3 and 4 for the column coater. Nevertheless, in spite of these differences in gloss and overall appearance, the coated tablets produced by either the coating column or the coating pan were of acceptable quality.

### EXAMPLE 11

### Plasdone K-29/32-Based Film Coating Kit using a Column Coater

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 3.00 | 42.86 | 3.00 | 100.00 | 100.00 | 100.00 |
| (hydrolyzed | | | | | | |
| starch product) | | | | | | |
| Plasdone K- | 3.00 | 42.86 | 3.00 | 100.00 | 100.00 | 100.00 |
| 29/32 (coating | | | | | | |
| resin) | | | | | | |
| PEG 3350 | 1.00 | 14.29 | 1.00 | 100.00 | 33.33 | 33.33 |
| (plasticizer) | | | | | | |
| Total Blend | 7.00 | 100.0 | 7.00 | 100.00 | 100.00 | — |

Procedure: The Maltrin 150 (hydrolyzed starch product, maltodextrin with a dextrose equivalent of between 15 and 17) (source, Grain Processing Corporation (GPC) of Muscatine, Iowa), Plasdone K-29/32 (N-vinyl-2-pyrrolidone, povidone, 44,000 to 54,000 molecular weight) (source, ISP Corporation of Wayne, N.J.), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson of Norristown, Pa.)

Deionized Water

Coating composition:

| Component | Gm/batch | % w/w | Gm solid/batch | % solid |
|---|---|---|---|---|
| Dry Mix | 7.00 | 3.50 | 7.00 | 100.00 |
| Marcoat 125 | 12.00 | 6.00 | 3.00 | 25.00 |
| (aqueous shellac) | | | | |
| Deionized water | 181.00 | 90.50 | 0.00 | 0.00 |
| Total Coating | 200.00 | 100.0 | 10.00 | 5.00 |
| System | | | | |

Procedure: The coating composition was prepared as in Example 1.

Coating process:

To test the coating composition for gloss and overall characteristics, 400 grams of placebo tablets were placed in a column coater and 107 grams of the coating material was applied, which corresponds to a weight gain of 1%. The coated tablets were tested for gloss and surface characteristics. The results are illustrated in Table 11.

TABLE 11
Coating Column Tablet Characteristics

| Weight Gain | Tricor Gloss | Orange Peel | | | |
|---|---|---|---|---|---|
| (%) | Reading | Frequency | Amplitude | Rash | Overall |
| 0.25 | 250 | 0.996 | 24500 | 1 | 1 |
| 0.50 | 249 | 0.995 | 22361 | 1 | 1 |
| 0.75 | 244 | 0.996 | 25200 | 1 | 1 |
| 1.00 | 249 | 0.997 | 25873 | 1 | 1 |

The above coating systems were found to be easy to work with, both in terms of time to prepare and viscosity. Example 12 and Reference Example 1 illustrate the different viscosities of the coating system and a prior art coating system.

### EXAMPLE 12

The coating system of Example 5 was repeated with proportionally increased amounts to make a 138.84 gram batch instead of the 46.28 gram batch. To make the batch, a beaker was filled with deionized water and the dry mix component was added with stirring. It took approximately 4 minutes and 50 seconds for the dry mix component to go into solution. The Marcoat 125 was added to the solution with stirring. It took approximately 2 minutes for the Marcoat 125 to go into solution. Therefore, the total time required to make a coating solution with 138 grams of dry mix component was approximately 7 minutes.

### REFERENCE EXAMPLE 1

The coating system described in Example 7 of Porter (U.S. Pat. No. 4,725,441) was generally followed, with the exception that 10% of PEG 3350 was used herein rather than 5% as disclosed by Porter, to make a coating suspension for purposes of measuring viscosity.

| Component | gm/batch | % w/w |
|---|---|---|
| Maltrin 150 (hydrolyzed starch product) | 105.00 | 70.00 |
| Propylene glycol alginate | 15.00 | 10.00 |
| PEG 400 (plasticizer) | 15.00 | 10.00 |
| PEG 3350 (plasticizer) | 15.00 | 10.00 |
| Total Blend | 150.00 | 100.0 |

Procedure:

1. 1350 grams of deionized water was measured out into a glass beaker.

2. The propylene glycol alginate was added to the deionized water.

3. The solution was heated to approximately 60° C to 70° C. and stirred until the propylene glycol alginate went into solution. This took approximately 35 minutes and one lump of the material still remained undissolved.

4. The remaining ingredients were added. This took approximately 10 minutes.

5. The solution was allowed to cool.

The coating solutions of Example 12 and Reference Example 1 were tested for viscosity at 25° C. The coating solution of Example 9 had viscosity measurements that ranged between 244.8 cP and 251.0 cP. The coating solution of Reference Example 1 has viscosity measurements that ranged between 440.0 cP and 500.0 cP. It is the inventors' experience and belief that such a high viscosity as obtained in Reference Example 1 is unsuitable for industrial scale application. With too high of a viscosity, the coating solution may be difficult to mix and prepare, may clog parts of the coating equipment, and may, in general, be too difficult to apply as a coating. It is the inventors' experience and belief that a viscosity of less than about 400 cP measured at 25° C. will result in a coating solution that is suitable for industrial scale application. And in particular, a viscosity of less than about 300 cP measured at 25° C. will result in a coating solution that is suitable for industrial scale application.

To determine the stability of a typical formulation, the inventors tested the settling characteristics of a formulation over five months.

### EXAMPLE 13

### Stability Testing for 20% Solids Formulation

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 20.00 | 44.44 | 140.00 | 100.00 | 100.00 | 100.00 |
| (hydrolyzed | | | | | | |
| starch product) | | | | | | |
| Plasdone | 140.00 | 44.44 | 140.00 | 100.00 | 100.00 | 100.00 |
| K-29-32 | | | | | | |
| (coating | | | | | | |
| resin) | | | | | | |
| PS80 | 7.00 | 2.22 | 7.00 | 100.00 | 5.00 | 5.00 |
| (plasticizer) | | | | | | |
| PEG 3350 | 28.00 | 8.89 | 28.00 | 100.00 | 20.00 | 20.00 |
| (plasticizer) | | | | | | |
| Total Blend | 315.00 | 100.0 | 315.00 | 100.00 | 100.00 | — |

Procedure: The Maltrin 150 (hydrolyzed starch product, maltodextrin with a dextrose equivalent of between 15 and 17) (source, Grain Processing Corporation (GPC) of Muscatine, Iowa), plasdone K-29-32 (N-vinyl-2-pyrrolidone, povidone, 44,000 to 54,000 molecular weight) (source, ISP Corporation of Wayne, N.J.), polysorbate 80 (PS80) (source, Unigema, B-Everberg), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson Resources, Norristown, Pa.)

Deionized Water

Coating composition

| Component | Gm/batch | % w/w | Gm solid/batch | % solid |
|---|---|---|---|---|
| Dry Mix | 383.50 | 15.34 | 383.50 | 100.00 |
| Marcoat 125 | 465.75 | 18.63 | 116.44 | 25.00 |
| (aqueous shellac) | | | | |
| Deionized water | 1650.75 | 66.03 | 0.00 | 0.00 |
| Total Coating | 2500.00 | 100.0 | 499.94 | 20.00 |
| System | | | | |

Procedure: The dry mix component and deionized water were blended together for approximately fifteen minutes until a clear solution was formed. The Marcoat 125 was then added and mixed for approximately ten minutes to form a clear, amber colored solution. The coating solution was divided into two containers and stored for five months. One container was to be examined without shaking and a second container was to be shaken if separation took place, and then one hour later examined. The container that was not to be shaken was found to free of settling for five month. The other container was found to have slight settling at four months. The container was shaken and the settling was corrected. At five months, the container was free of settling.

### EXAMPLE 14

### Stability Testing for 30% Solids Formulation

Part A. Dry Mix Component

| | | | gm solid/ | | % solid | |
|---|---|---|---|---|---|---|
| Component | gm/batch | % w/w | batch | % solids | w/w | % polymer |
| Maltrin 150 | 220.00 | 44.44 | 220.00 | 100.00 | 100.00 | 100.00 |
| (hydrolyzed | | | | | | |
| starch | | | | | | |
| product) | | | | | | |
| Plasdone K- | 220.00 | 44.44 | 220.00 | 100.00 | 100.00 | 100.00 |
| 29-32 (coating | | | | | | |
| resin) | | | | | | |
| PS80 | 11.00 | 2.22 | 11.00 | 100.00 | 5.00 | 5.00 |
| (plasticizer) | | | | | | |
| PEG 3350 | 44.00 | 8.89 | 44.00 | 100.00 | 20.00 | 20.00 |
| (plasticizer) | | | | | | |
| Total Blend | 495.00 | 100.0 | 495.00 | 100.00 | 100.00 | — |

Procedure: The Maltrin 150 (hydrolyzed starch product, maltodextrin with a dextrose equivalent of between 15 and 17) (source, Grain Processing Corporation (GPC) of Muscatine, Iowa), plasdone K-29-32 (N-vinyl-2-pyrrolidone, povidone, 44,000-54,000 molecular weight) (source, ISP Corporation of Wayne, N.J.), polysorbate 80 (PS80) (source, Unigema, B-Everberg), and polyethylene glycol (PEG) 3350 (source, Clariant Corporation of Somerville, N.J.) were mixed, packaged and stored as in Example 1.

Part B. Liquid Components

Marcoat 125 (shellac, 25% solids) (source, Emerson Resources, Norristown, Pa.)

Deionized Water

Coating composition

| Component | Gm/batch | % w/w | Gm solid/batch | % solid |
|---|---|---|---|---|
| Dry Mix | 575.25 | 23.01 | 575.25 | 100.00 |
| Marcoat 125 | 898.75 | 27.95 | 174.69 | 25.00 |
| (aqueous shellac) | | | | |
| Deionized water | 1226.00 | 49.04 | 0.00 | 0.00 |
| Total Coating | 2500.00 | 100.0 | 749.94 | 30.00 |
| System | | | | |

Procedure: The dry mix component and deionized water were blended together for approximately fifteen minutes until a clear solution was formed. The Marcoat 125 was then added and mixed for approximately ten minutes to form a clear, amber colored solution. The coating solution was divided into two containers and stored for five months. One container was to be examined without shaking and a second container was to be shaken if separation took place, and then one hour later examined. Both containers were found to free of settling for five months although a gelatinous mass was found to be forming by the fifth month.

### EXAMPLE 15

### EM 273, 11.1% Solids

| | Component | Weight | Percentage |
|---|---|---|---|
| Component | Category | (grams) | (w/w) |
| Marcoat 125 | Shellac | 10.0* | 12.35 |
| (aqueous solution containing 25% | | | |
| shellac) | | | |
| Maltrin 150 | Hydrolyzed | 4.5 | 5.55 |
| (maltodextrin having a DE of 13-17) | Starch | | |
| | Product | | |
| Plasdone S630 | Film | 0.5 | 0.61 |
| (60:40 copolymer of vinyl | Coating | | |
| pyrrolidone and vinyl acetate) | Resin | | |
| PEG 3350 | Plasticizer | 1.5* | 1.85 |
| (polyethylene glycol, 50% aqueous | | | |
| solution) | | | |
| Water | | 64.5 | 79.63 |
| Total | | 81.0 | 100 |

*this weight refers to the weight of the solution itself and corresponds to 2.5 grams of shellac solids and 0.75 grams PEG 3350, respectively.

The coating formulation was prepared as in Example 1 and applied using a column coater. The gloss was measured using a Tricor Surface Analysis System and the resulting gloss was observed likely to be greater than 250.

### EXAMPLE 16

### EM 274,10.9% Solids

| | Component | Weight | Percentage |
|---|---|---|---|
| Component | Category | (grams) | (w/w) |
| Marcoat 125 | Shellac | 10.0* | 12.12 |
| (aqueous solution containing 25% | | | |
| shellac) | | | |
| Maltrin 150 | Hydrolyzed | 2.5 | 3.03 |
| (maltodextrin having a DE of 13-17) | Starch | | |
| | Product | | |
| PVP K29-32 (polyvinyl pyrrolidone) | Film | 2.5 | 3.03 |
| | Coating | | |
| | Resin | | |
| PEG 3350 | Plasticizer | 1.5* | 1.82 |
| (polyethylene glycol, 50% aqueous | | | |
| solution) | | | |
| Water | | 66.0 | 80.00 |
| Total | | 82.5 | 100 |

*this weight refers to the weight of the solution itself and corresponds to 2.5 grams of shellac solids and 0.75 grams PEG 3350, respectively.

The coating formulation was prepared as in Example 1 and applied using a column coater. The gloss was measured using a Tricor Surface Analysis System and, for a 1% weight gain, the gloss was found to be 243.

Table 17 is a chart listing the Tricor gloss readings for Examples 1-11.

TABLE 17
Tricor Gloss Readings

| Wt. | | | | | | | | | | | |
|---|---|---|---|---|---|---|---|---|---|---|---|
| gain | | | | | | | | | | | |
| (%) | Ex. 1 | Ex. 2 | Ex. 3 | Ex. 4 | Ex. 5 | Ex. 6 | Ex. 7 | Ex. 8 | Ex. 9 | Ex. 10 | Ex. 11 |
| 0.25 | 213 | 230 | 222 | 224 | 235 | 230 | 237 | 240 | 226 | 250 | 250 |
| 0.5 | 218 | 221 | 215 | 231 | 238 | 238 | 250 | 245 | 234 | 245 | 249 |
| 0.75 | 223 | 226 | 216 | 226 | 239 | 237 | 257 | 242 | 228 | 230 | 244 |
| 1.00 | 221 | 214 | 215 | 237 | 247 | 260 | 261 | 255 | 242 | 224 | 249 |

The coating composition of the above examples or the other coating composition examples described herein may be applied to tablets which have been coated or tablets which are uncoated. The application may be made in a fluid bed coater or a standard side vented pan. Using standard spray guns and coating parameters, a glossy film may be easily applied. As little as 0.25% weight gain may supply the gloss desired on coated tablets. More coating may be applied, especially if a seal on an uncoated tablet is required. Not only tablets may be coated, but granules, beads, gum pieces, candies and nuts may all be coated. Various pharmaceutical products, nutraceutical products and food supplements on the market today may be top coated with this film coating composition in the absence of pigments to provide a clear gloss that enhances the appearance of the product.

It should be noted that the above examples involve the use of approximately 5% solids. Most of these coatings give a good gloss at 5% solids, however, this system may be diluted to a lower percentage of solids if desired, even 2.5% solids or lower. Any of the formulation examples illustrated herein may be diluted to spray at lower solids or concentrate their spray at higher solids, up to 15% solids and possibly even 20% solids in some cases. In general the formulations may be adjusted to concentrations of about 5% to about 15% solids and readily coated to give a very glossy finish.

Flavors and sweeteners may be added to the system to enhance the taste and mouth feel on the coating without diminishing the gloss.

In accordance with the present invention, it has been found that the coating solution may be stabilized by the addition of salts of ethylene diamine tetraacetic acid (EDTA) to the solution to prevent the degradation. The salts of EDTA may be disodium, trisodium and tetrasodium salts of EDTA and mixtures of disodium, trisodium and tetrasodium salts of EDTA. Mixtures of disodium, trisodium and tetrasodium salts of EDTA stabilize the solution at about 1% of the solution. The addition of salts of EDTA permits the preparation of solutions with 10% to 40% solids. These solutions will have a shelf life of three or more months. This will permit the preparation of high gloss coating solutions for the commercial market, including some storage, shipping time and storage and application time of the coater, as well as other applications and processes which require time after manufacture of the coating composition.

While several particular forms of the invention have been illustrated and described, it will be apparent that various modifications and combinations of the invention detailed in the text can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited, except as by the appended claims.

What is claimed is:

1. A film coating composition comprising:
a shellac in an aqueous solution;
a hydrolyzed starch product;
optionally, a plasticizer; and
a coating resin comprising one or more of polyvinyl pyrrolidone, polyvinyl alcohol, and poly(vinyl pyrrolidone-vinyl acetate), wherein the shellac, hydrolyzed starch product, plasticizer and coating resin comprise the solids in the composition.

2. The film coating composition of claim 1, wherein the film coating composition is applied to an edible surface to provide a Tricor reading of gloss that is greater than 200.

3. The film coating composition of claim 1, wherein the film coating composition is applied to an edible surface to provide a Tricor reading of gloss that is between 200 and 250.

4. The film coating composition of claim 1, wherein the coating resin is present in an amount of between approximately 10% and 30% (w/w) of the solids in the composition.

5. The film coating composition of claim 1, wherein the coating resin comprises polyvinylpyrrolidone.

6. The film coating composition of claim 1, wherein the coating resin comprises poly(vinyl pyrrolidone-vinyl acetate).

7. The film coating composition of claim 1, wherein the shellac comprises between about 10% and 30% (w/w) of the solids in the composition.

8. The film coating composition of claim 1, wherein the hydrolyzed starch product comprises between about 20% and 60% (w/w) of the composition.

9. The film coating composition of claim 1, wherein the hydrolyzed starch product comprises one or both of a maltodextrin and a corn syrup solid.

10. The film coating composition of claim 1, wherein the hydrolyzed starch product has a dextrose equivalent value of between about 4 and 25.

11. The film coating composition of claim 1, wherein the hydrolyzed starch product has a dextrose equivalent value of between 15 and 20.

12. The film coating composition of claim 1, wherein the plasticizer comprises between about 5% and 25% (w/w) of the solids in the composition.

13. The film coating composition of claim 1, wherein the plasticizer comprises one or more of triacetin (TA), triethylcitrate (TEC), polyethylene glycol (PEG), propylene glycol (PG), glycerine, glycerol monostearate (GMS), diacetylated monoglyceride (DAM) and polysorbate.

14. The film coating composition of claim 13, wherein the polyethylene glycol comprises a molecular weight of 200 to 8000.

15. The film coating composition of claim 13, wherein the polysorbate comprises one or more of polysorbate 60 and polysorbate 80.

16. The film coating composition of claim 1, wherein the composition is applied to a pharmaceutical product, nutraceutical product, or food product.

17. The film coating composition of claim 1, wherein the composition has a viscosity of less than about 400 cP measured at 25° C.

18. The film coating composition of claim 1, wherein the composition has a viscosity of less than about 300 cP measured at 25° C.

19. A multi-component kit for formulating a film coating composition, the kit comprising a dry mix component and an aqueous shellac, the dry mix component comprising:
a hydrolyzed starch product;
optionally, a plasticizer; and
a coating resin comprising one or more of polyvinyl pyrrolidone, polyvinyl alcohol, and poly(vinyl pyrrolidone-vinyl acetate), wherein the shellac, hydrolyzed starch product, plasticizer and coating resin comprise the solids in the composition.

20. The kit of claim 19, wherein the film coating composition is applied to an edible surface to provide a Tricor reading of gloss that is greater than 200.

21. The kit of claim 19, wherein the film coating composition is applied to an edible surface to provide a Tricor reading of gloss that is between 200 and 250.

22. The kit of claim 19, wherein the coating resin is present in an amount of between approximately 10% and 30% (w/w) of the solids in the composition.

23. The kit of claim 19, wherein the coating resin comprises polyvinylpyrrolidone.

24. The kit of claim 19, wherein the coating resin comprises poly(vinyl pyrrolidone-vinyl acetate).

25. The kit of claim 19, wherein the shellac comprises between about 10% and 30% (w/w) of the solids in the composition.

26. The kit of claim 19, wherein the hydrolyzed starch product comprises between about 20% and 60% (w/w) of the solids in the composition.

27. The kit of claim 19, wherein the hydrolyzed starch product comprises one or both of a maltodextrin and a corn syrup solid.

28. The kit of claim 19, wherein the plasticizer comprises between about 5% and 25% (w/w) of the solids in the composition.

29. The kit of claim 19, wherein the plasticizer comprises one or more of triacetin (TA), triethylcitrate (TEC), polyethylene glycol (PEG), propylene glycol (PG), glycerine, glycerol monostearate (GMS), diacetylated monoglyceride (DAM) and polysorbate.

30. The kit of claim 29, wherein the polyethylene glycol comprises a molecular weight of 200 to 8000.

31. The kit of claim 29, wherein the polysorbate comprises one or more of polysorbate 60 and polysorbate 80.

32. The kit of claim 19, wherein the composition is applied to a pharmaceutical product, nutraceutical product, or food product.

33. The kit of claim 19, wherein the composition has a viscosity of less than about 400 cP measured at 25° C.

34. A method of making a coating mixture, the method comprising:
providing a dry mix component, the dry mix component comprising a hydrolyzed starch product, an optional plasticizer, and a coating resin comprising one or more of polyvinyl pyrrolidone, polyvinyl alcohol, and poly(vinyl pyrrolidone-vinyl acetate);
providing an aqueous shellac;
mixing the dry mix component with water to form an aqueous mixture; and
mixing the aqueous mixture with the aqueous shellac to form the coating mixture, wherein the shellac, hydrolyzed starch product, plasticizer and coating resin comprise the solids in the composition.

35. The method of claim 34, further comprising a step of separately providing the hydrolyzed starch product, the plasticizer and the coating resin, and mixing to form the dry mix component.

36. The method of claim 34, wherein the composition has a viscosity of less than about 400 cP measured at 25° C.

37. The film coating composition of claim 34, wherein the composition has a viscosity of less than about 300 cP measured at 25° C.
